(19) United States
(12) Patent Application Publication
(10) Pub. No.: US 2023/0181652 A1
(43) Pub. Date: Jun. 15, 2023

(54) COMPOSITION FOR ENHANCING COGNITIVE ABILITIES AND USE THEREOF

(71) Applicant: NatureWise Biotech & Medicals Corporation, Taipei City (TW)

(72) Inventors: Chia-Chung HOU, Taipei City (TW); Yi-Ju LIANG, Taipei City (TW); Chung-Yang HUANG, Taipei City (TW)

(73) Assignee: NatureWise Biotech & Medicals Corporation, Taipei City (TW)

(21) Appl. No.: 18/080,825

(22) Filed: Dec. 14, 2022

(30) Foreign Application Priority Data
Dec. 14, 2021 (TW) 110146781

(51) Int. Cl. A61K 35/644; A61K 36/48; A61P 25/28

(52) U.S. Cl. CPC A61K 35/644; A61K 36/48; A61P 25/28

(57) ABSTRACT

Provided is a composition for enhancing cognitive abilities and use thereof; wherein the composition consists essentially of royal jelly, Taiwan green propolis and soybean lecithin; and the cognitive abilities comprise memory, judgment, intuition, calculation, and reaction abilities.

### TECHNOLOGY FIELD

The present invention relates to a composition for improving cognitive abilities and use thereof.

### BACKGROUND OF THE INVENTION

Sufficient nutrients are the basis for maintaining the normal metabolism of the central nervous system. These nutrients, including amino acids, multiple unsaturated fatty acids, vitamins, inorganic salts, glucose, etc., can protect the brain and hippocampus and promote the functional recovery of nerve cells, which facilitate the learning and memory abilities. With the increase of age, brain will have the problem of cognitive decline, and insufficient intake of nutrients due to the pressure of life and job will also cause cognitive decline. Currently, it is found that the decline in cognitive abilities of children and teenagers, such as declines in memory and reaction abilities, is mostly caused by stress and malnutrition. Physical lesions in the brain could also cause cognitive decline. For example, certain chronic diseases, such as endocrine dysfunction, malnutrition, chronic poisoning, blood circulation disorders and cervical spondylosis, could damage the brain and cause cognitive decline. For those people who need to improve memory and prevent cognitive decline, they take some health foods in addition to nutrients from their daily diet.

In the past, it is mentioned in many literatures that green propolis has many physiological effects, such as antibacterial activity, anti-inflammation, antioxidation, improvement of type 2 diabetes, inhibition of obesity, neuroprotective effects and other biological activities. Recently, it was disclosed that Brazilian propolis is effective in preventing cognitive abilities decline in seniors at high altitudes (Journal of Alzheimer's Disease, vol. 63, no. 2, pp. 551-560, 2018).

It is still desirable to develop a food supplement to improve cognitive abilities in humans.

### SUMMARY OF THE INVENTION

It is unexpectedly found in the subject application that a new composition is effective as a food supplement in maintaining the survival of neural stem cells, inducing neuron differentiation, promoting the proliferation of nerve fibers, preventing neuron death caused by free radical attacking nerve cells and improving the cognitive abilities of brain in a subject, particularly in humans.

In one aspect, the present invention provides a composition for improving cognitive abilities, which consists essentially of royal jelly, Taiwan green propolis extract and soybean lecithin, as active components.

In one embodiment of the present invention, the composition consists of royal jelly, Taiwan green propolis extract and soybean lecithin, as active components.

In one example of the present invention, the composition further comprises auxiliary ingredients: Gastrodia elata extract, black soybean powder, and soybean germ powder.

In one example of the present invention, the royal jelly contains decenoic acid in the content of more than 3% by the weight.

In one example of the present invention, the Taiwan green propolis extract contains isoprenoid flavonoids in the contents of more than 8% by the weight.

In one preferred example of the present invention, the soybean lecithin contains phosphatidylserine (PS) in the content of more than 70% by the weight.

According to the present invention, the composition comprises more than 1% of decenoic acid, more than 2% of isoprenoid flavonoids and more than 1% of phosphatidylserine by the weight of the composition.

In one embodiment of the present invention, the composition comprises 135 to 750 grams of royal jelly, 100 to 600 grams of Taiwan green propolis extract, 10 to 70 grams of soybean lecithin, 18 to 240 grams of black soybean powder, 15 to 125 grams of Gastrodia elata extract and 55 to 325 grams of soybean germ powder per 1000 grams of the composition.

In one embodiment of the present invention, the composition consists of 270 to 500 grams of royal jelly, 200 to 400 grams of Taiwan green propolis extract, 20 to 45 grams of soybean lecithin, 36 to 160 grams of black soybean powder, 30 to 82 grams of Gastrodia elata extract and 115 to 215 grams of soybean germ powder, as active components, per 1000 grams of the composition.

In one embodiment of the present invention, the composition consists of 338 to 420 grams of royal jelly, 253 to 315 grams of Taiwan green propolis extract, 26 to 36 grams of soybean lecithin, 45 to 134 grams of black soybean powder, 41 to 68 grams of Gastrodia elata extract and 143 to 178 grams of soybean germ powder, as active components, per 1000 grams of the composition.

According to the present invention, the composition is in the form of a nutriment supplement, or a food supplement as a healthcare food or a health food.

According to the present invention, the composition can be prepared as a medicated food.

According to the present invention, the composition can be prepared as a medicine.

As used herein, the term “cognitive abilities” refers to memory, and the abilities of judgment, intuition, calculation and reaction.

According to the present invention, the composition further provides the effects on resistance of memory degradation, prevention of dementia, and/or decline or damages of brain cell functions, activation of brain cells, improving memory, learning ability, and attention of a subject, and the composition is helpful for clear thinking and keeps the mind sharp, and it can be absorbed well.

It is to be understood that both the foregoing general description and the following embodiments are exemplary and illustrative only and are not intended to limit the present invention.

### BRIEF DESCRIPTION OF THE DRAWINGS

The foregoing summary of the invention and the following detailed descriptions of the present invention will be better understood when read in conjunction with the accompanying drawing. In the drawing:

FIG. 1 shows that there were 11, 14 and 13 people in the placebo group, the green propolis group and the test group respectively. The effects of different nutritional supplements on brain functional indices were discussed, and the differences between groups were analyzed using one-way variance. The label of different letters (a, b) on data of each group indicated statistically significant difference (p<0.05); and dependent-sample t test was used to evaluate statistical results of the pre- and post-tests within groups, *, p<0.05.

### DETAILED DESCRIPTION OF THE INVENTION

In addition to the above contents, the present invention will be further described with reference to the following exemplary embodiments. However, it should not be construed that the contents of the present invention are limited to the following embodiments, but all inventions based on the contents of the present invention belong to the scope of the present invention.

Unless otherwise defined, all technical and scientific terms used herein have the same meaning as commonly understood by people having ordinary skill in the art to which this invention belongs.

As used herein, the singular forms “a,” “an,” and “the” include plural referents unless the context clearly dictates otherwise. Thus, for example, reference to “a sample” includes a plurality of such samples and equivalents thereof known to those skilled in the art.

The term “individual” or “subject” as used herein includes humans and non-human animals, such as pet animals (e.g., dogs, cats, etc.), farm animals (e.g., cows, sheep, pigs, horses, etc.), or laboratory animals (e.g., rats, mice, guinea pigs, etc.). In the present invention, the term “individual” or “subject” mainly refers to a human.

The term “improving” or “improvement” as used herein refers to the application or administration of the composition of the present invention to a subject in need for the purpose of enhancing, promoting, enhancing, enhancing, strengthening, strengthening, increasing, changing, improving, ameliorating or affecting the performance of the functions. Specifically, it refers to an increase in the measured score of cognitive ability of a subject in the present invention.

According to the present invention, the composition can be administered by various routes known in the art, preferably orally. It can be prepared as a health food or a healthcare food according to commonly known or standard methods to those skilled in the art.

According to an embodiment of the present invention, the composition of the present invention can be formulated together with a pharmaceutically acceptable carrier to form a pharmaceutical composition in a suitable form (e.g., an oral medicament). Depending on the mode of administration, the pharmaceutical composition of the present invention may comprise from 0.1% to 100% by weight of the composition of the present invention, wherein the weight percentages are calculated based on the total weight of the composition. A “pharmaceutically acceptable carrier” is non-toxic to a subject at the dosage and concentration employed, and is compatible with any other ingredient of any form of formulation of the composition of the present invention.

According to the present invention, the composition may be administered by various routes a known in the art, for example, via blood injection or subcutaneous injection, or being delivered directly from the respiratory tract to the trachea, or the lung, e.g., by direct nasal inhalation; or being administered by bronchoscopy through the nose or mouth to the trachea, or being administered by tracheotomy.

In one aspect, the present invention provides a composition for improving cognitive abilities, wherein the composition consists essentially of royal jelly, Taiwan green propolis extract and soybean lecithin.

According to the present invention, the composition further comprises auxiliary ingredients: Gastrodia elata extract, black soybean powder, and soybean germ powder.

According to the present invention, the royal jelly contains decenoic acid in in the content of more than 3%.

According to the present invention, the Taiwan green propolis extract contains isoprenoid flavonoids in the content of more than 8%.

According to the present invention, the soybean lecithin contains phosphatidylserine in the content of more than 70%.

According to the present invention, the composition comprises more than 1% of decenoic acid, more than 2% of isoprenoid flavonoids and more than 1% of phosphatidylserine.

In one embodiment of the present invention, the composition comprises 135 to 750 grams of royal jelly, 100 to 600 grams of Taiwan green propolis extract, 10 to 70 grams of soybean lecithin, 18 to 240 grams of black soybean powder, 15 to 125 grams of Gastrodia elata extract and 55 to 325 grams of soybean germ powder per 1000 grams by the total weight of the composition. Preferably, the composition comprises 270 to 500 grams of royal jelly, 200 to 400 grams of Taiwan green propolis extract, 20 to 45 grams of soybean lecithin, 36 to 160 grams of black soybean powder, 30 to 82 grams of Gastrodia elata extract and 115 to 215 grams of soybean germ powder per 1000 grams of the composition. Most preferably, the composition comprises 338 to 420 grams of royal jelly, 253 to 315 grams of Taiwan green propolis extract, 26 to 36 grams of soybean lecithin, 45 to 134 grams of black soybean powder, 41 to 68 grams of Gastrodia elata extract and 143 to 178 grams of soybean germ powder per 1000 grams of the composition.

According to the present invention, the composition is in the form of a nutriment supplement, a health food or a healthcare food.

According to the present invention, the composition of the present invention further provides the effects on resistance of memory degradation, prevention of dementia, and/or decline or damages of brain cell functions, activation of brain cells, improving memory, learning ability, and attention of a subject, and the composition is helpful for clear thinking and keeps the mind sharp, as well as it can be absorbed well.

According to the present invention, the composition can be prepared as a medicated food or a drug.

According to the present invention, the composition of the present invention can improve cognitive abilities, including memory, judgment, intuition, calculation and reaction abilities.

According to an embodiment of the present invention, the composition of the present invention can effectively improve the performance of a subject on the MoCA scale.

In one embodiment, the composition of the present invention can effectively improve a subject's cognitive abilities, which comprise memory, judgment, intuition, calculation and reaction abilities, and in particular improve an elder's cognitive abilities, especially the intuition, calculation and reaction abilities.

In another aspect, the present invention provides a use of the composition for preparing a drug, a health food, or a healthcare food for improving cognitive abilities of a subject, especially improving memory, intuition, calculation and reaction abilities.

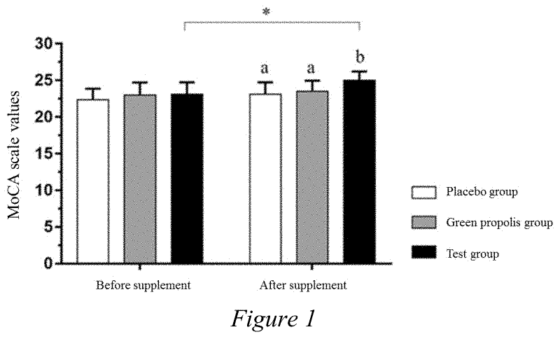

According to the embodiment of the present invention, as shown in FIG. 1, after supplementing the composition of the present invention for two consecutive months, there exists significant difference between groups (Post-MoCA) (F(2,35)=6.08, p=0.005), showing that the test group is significantly higher than the placebo group and green propolis group (p=0.003; p=0.01), but the pre- and post-test results have no significant difference within the placebo group and within the green propolis group. In addition, it is shown that the test group taking the composition of the present invention has significantly higher post-test values in memory, intuition, calculation and reaction capability indexes than the pre-test values (p=0.002; p=0.017; p=0.047; p=0.009), and has significant positive benefits in intuition, calculation and reaction capabilities. The above test results show that the composition of the present invention provides a better synergistic effect than the green propolis group, and in particular improves the cognitive abilities, especially the intuitive, calculation and reaction capabilities, of the elderly.

### EXAMPLES

Material and Methods

I. Preparation Method of the Composition of the Present Invention

1. Preparation of Taiwan Green Propolis Extract

After the Taiwan green bee propolis block is tested and identified, first remove foreign matter and impurities, extract Taiwan green propolis extract with edible alcohol, add corn starch granulation after concentrating, and obtain Taiwan green propolis extract powder.

2. Preparation of Royal Jelly

After royal jelly raw material is tested and identified, first remove foreign matters and impurities, add corn starch for milling and mixing, and then remove moisture through freeze-vacuum drying to obtain the royal jelly powder.

3. Preparation of the Composition of the Present Invention

The royal jelly powder, Taiwan green propolis extract powder and soybean lecithin powder were milled and stirred uniformly at a ratio of 5:4:1, and adding an appropriate amount of soybean germ powder, Gastrodia elata extract and black soybean powder, and then adding oxidized starch as filler and magnesium stearate as dispersant to make hard capsules.

II. MoCA Scale and “Everyday Brain Exercise” Application

Functional assessment method of MoCA Montreal Intelligence Test Scale: Before the intervention, the oral and tutoring were completed according to the scale, and the visual space, naming, concentration, language, abstract concept, delayed memory and orientation were tested for an expected time of 30 minutes and will be retested after two consecutive months of experimental intervention. Functional assessment methods of “Everyday Brain Exercise” application: after completing the MoCA scale, the observation, memory, judgment, intuition, calculation and reaction capabilities were evaluated using tablet computer.

III. Research Objects

Number of recruitments: 45 people; inclusion criteria: score greater than 20 points in the MoCA test; age: 55 to 85 years old; and 38 people completed the test. Exclusion criteria and grouping method: a. those with visual less than 0.5 after correction, b. stroke patients, c. allergic to propolis or bee products, d. adults with no mental capacity or MoCA test scores below 20. Note: In order to avoid people who were allergic to propolis without testing their allergens, they would be preferentially excluded by interviews: 1. those who had been allergic to honey, pollen, royal jelly or aspirin, 2. those with asthma, 3. those who has blood coagulation disorder (hemophilia) or those who was taking anticoagulants.

After the MoCA functional assessment was performed before the intervention, the tested people were evenly grouped into (A) placebo group (11 people): corn starch capsules, (B) control group (14 persons): green propolis group and (C) test group (13 persons): capsules of the present invention according to the pre-test analysis results, in which the order of scores was adopted.

Test Methods

Research Test Design or Implementation:

The test adopted the single-blind design, MoCA functional assessment was performed before the intervention, and according to the pre-test analysis scores, people were divided evenly into 3 groups, with 15 people in each group, in which the order of scores was adopted.

Placebo group: placebo capsules without Taiwan green propolis extract (containing corn starch), 2 capsules each time, 2-3 times a day.

Green propolis group: containing Taiwan green propolis extract and corn starch, 2 capsules each time, 2-3 times a day.

Test group: capsules of the composition of the present invention (royal jelly powder, Taiwan green propolis extract powder, specially selected soybean germ powder, calcium D-pantothenate, Gastrodia elata extract, refined black soybean powder, soybean lecithin powder (containing phosphatidylserine), 2 capsules each time, 2-3 times a day.

IV. Statistical Methods

The data statistics method determined the use of different statistical methods according to whether the data presented a normal distribution or not. In the parametric statistical method, dependent-sample t test, independent-sample t test and one-way analysis of variance were used to test whether there was a difference between different treatments (various sub-indices of Everyday Brain Exercise application and total integral score of MoCA scale). If the data cannot fit the normal distribution, the nonparametric statistical methods Wilcoxon and Kruskal-Wallis were used to analyze the differences within groups and between groups (each sub-indices of MoCA scale), and P<0.05 represented statistical significance.

### Example 1: Effect of the Composition of the Present Invention on Brain Functional Assessment

The brain functional assessment software application was used to evaluate six brain related function indices, such as observation, memory, judgment, intuition, calculation and reaction capabilities. According to MoCA divided groups, the subjects under test were given pre- and post-tests for the evaluation of differences within and between groups.

TABLE 1
Sub-indices of the application software APP

| | | |
|---|---|---|
| Observation | Memory | Judgement |

| | Pre | Post | Pre | Post | Pre | Post |
|---|---|---|---|---|---|---|
| Placebo group | 42 ± 13 | 38 ± 11 | 84 ± 31 | 76 ± 24^{a} | 50 ± 25 | 55 ± 23^{a} |
| Green propolis | 59 ± 28 | 62 ± 32 | 101 ± 38 | 112 ± 43*^{,b} | 64 ± 23 | 78 ± 33*^{,b} |
| group | | | | | | |
| Test group | 50 ± 22 | 57 ± 32 | 78 ± 31 | 102 ± 35*^{,b} | 58 ± 18 | 66 ± 15^{+,ab} |

| | Intuition | | Calculation | | Reaction | |
|---|---|---|---|---|---|---|
| | Pre | Post | Pre | Post | Pre | Post |
| Placebo group | 161 ± 134 | 236 ± 105 | 114 ± 63 | 128 ± 96 | 36 ± 19 | 38 ± 13 |
| Green propolis | 157 ± 72 | 224 ± 139 | 149 ± 87 | 146 ± 83 | 55 ± 43 | 55 ± 41 |
| group | | | | | | |
| Test group | 153 ± 106 | 278 ± 121* | 146 ± 87 | 156 ± 85* | 36 ± 13 | 41 ± 11* |

There were 11, 14 and 13 people in the placebo group, the green propolis group and the test group respectively. The effects of different nutritional supplements on brain functional indices were discussed, and the differences between groups were analyzed using one-way analysis of variance. The label of different alphabet (a, b) on data of each group indicated statistically significant difference (p<0.05); and dependent-sample t test was used to evaluate statistical results of the pre- and post-tests within groups, *, p<0.05. (+, p=0.054)

As shown in Table 1, there was no significant difference between groups in all the six pre-test indices (F(2,35)=0.81; F(2,35)=1.76; F(2,35)=0.91; F(2,35)=0.016; F(2,35)=0.69; F(2,35)=1.82, both p>0.05), showing that there was no significant difference existing in brain functional indices after grouping and before supplementation. After two consecutive months of supplementation, there was no significant difference existing between groups with respect to the four indices of observation, intuition, calculation and reaction capabilities (F(2,35)=1.65; F(2,35)=0.69; F(2,35)=0.23; F(2,35)=0.93, both p>0.05), but there were significant differences between groups in memory and judgment indices (F(2,35)=6.33; F(2),35)=3.48, p<0.05). In the post-memory test, the green propolis group and the test group were significantly higher than the placebo group (p=0.023, p=0.001), but there was no significant difference between the green propolis group and the test group (p=0.263); in the post-judgment test, the green propolis group was significantly higher than the placebo group (p=0.033), and there was no significant difference between the test group, the green propolis group and the placebo group (p=0.28; p=0.255).

Regarding pre- and post-test part within groups, all the values of post-tests of the green propolis group were significantly higher than that of pre-tests in memory and judgment indices (p=0.03; p=0.047). As for the test group, all the values of post-tests were significantly higher than that of pre-tests in memory, intuition, calculation and reaction indices (p=0.002; p=0.017; p=0.047; p=0.009).

### Example 2: Effect of the Composition of the Present Invention on MoCA Scale Assessment

The experimental groups comprised placebo group, green propolis group and test group that took the composition of the present invention. The subjects who were subject to pre-test of MoCA scale and met the acceptance criteria of this experiment (greater than 20 points in the MoCA test) were divided into placebo group (corn starch capsules), green propolis group (green propolis capsules) and test group (test capsules) according to scores adopting S-type sorting (normal distribution). After two consecutive months of supplementation, single-blind design was adopted in the experiment and post-tests were carried out.

TABLE 2
Sub-indices of MoCA scale

| | Visual space | | Naming | | Concentration | | Language | |
|---|---|---|---|---|---|---|---|---|
| | Pre | Post | Pre | Post | Pre | Post | Pre | Post |
| Placebo group | 3.5 ± 1 | 3.5 ± 1^{a} | 2.9 ± 0.3 | 3.0 ± 0 | 6.0 ± 0.0 | 6.0 ± 0.0 | 2.5 ± 0.7 | 2.9 ± 0.3 |
| Green propolis | 4 ± 1 | 4.1 ± 0.8^{a} | 3.1 ± 0.8 | 3.0 ± 0 | 6.0 ± 0.0 | 5.8 ± 0.6 | 2.6 ± 0.6 | 2.7 ± 0.6 |
| group | | | | | | | | |
| Test group | 4.3 ± 0.6 | 4.5 ± 0.7^{b} | 3.1 ± 0.3 | 3.0 ± 0 | 5.8 ± 0.4 | 5.9 ± 0.3 | 2.5 ± 0.5 | 2.9 ± 0.3 |

| | Abstract concepts | | Delayed memory | | Orientation | |
|---|---|---|---|---|---|---|
| | Pre | Post | Pre | Post | Pre | Post |
| Placebo group | 0.9 ± 0.5 | 1.2 ± 0.8 | 0.6 ± 1.0 | 0.5 ± 0.7^{a} | 5.8 ± 0.4 | 5.9 ± 0.3 |
| Green propolis | 0.9 ± 0.3 | 1.4 ± 0.6* | 0.4 ± 0.9 | 0.4 ± 0.6^{a} | 6.0 ± 0.0 | 5.8 ± 0.8 |
| group | | | | | | |
| Test group | 1.2 ± 0.6 | 1.6 ± 0.5* | 0.3 ± 0.6 | 1.0 ± 0.7^{b,}* | 5.8 ± 0.6 | 6.0 ± 0.0 |

There were 11, 14 and 13 people in the placebo group, green propolis group and test group respectively. The effects of different nutritional supplements on the sub-indices of MoCA scale were discussed, and the differences between groups were analyzed by nonparametric statistical method Kruskal-Wallis. The label of different alphabet (a, b) on data of each group indicated statistically significant difference (p<0.05); and statistical results of the pre- and post-tests within groups were evaluated by Wilcoxon method, *, p<0.05.

As shown in Table 2, in the grouping of pre-test normal distribution, there was no significant difference in MoCA scale indices between groups (F(2,35)=0.67, p=0.518), indicating that the groups were the same at the basal starting point before supplementation and there were no differences existing between groups. The groups were given supplementation for two consecutive months according to the experiment design. After two consecutive months of supplementation (FIG. 1), there were significant differences between groups (Post-MoCA) (F(2,35)=6.08, p=0.005). The differences between groups were further analyzed by Duncan post-test, showing that the test group was significantly higher than the placebo group and the green propolis group (p=0.003; p=0.01), but there was no significant difference between the placebo group and the green propolis group (p=0.485). The results within groups showed that the post-test of the test group was significantly higher than the pre-test as well: the MoCA scale value (p=0.006); but there was no significant difference within groups in the pre- and post-test results of the placebo group and the green propolis group (p=0.053 vs p=0.22).

Regarding the MoCA sub-index part, the post-test part of the visual space index showed that the test group was significantly higher than the placebo group and the green propolis group (p<0.05), but there was no significant difference between the placebo group and the green propolis group. As for the abstract concept index, both the post-tests of the green propolis group and the test group were significantly higher than the pre-tests (p<0.05), but there was no significant difference between groups. On the delayed memory index, besides the post-test index was significantly higher than the pre-test index (p<0.05) within the supplementary group of the test group, the post-test differences between groups of the test group were also significantly higher than that of the placebo group and the green propolis group (p<0.05).), but there was no significant difference between groups with respect to the placebo group and the green propolis group.

In view of the above, the test group that took the composition of the present invention had significantly improved benefits in the performance of memory and judgment capabilities (MoCA scale and application software), compared with the placebo group. In addition to the above indices, there were also significant and positive benefits in all the intuitive, calculation and reaction capabilities (application software).

All the patents and references cited in the specification are incorporated by reference in their entirety. In case of conflict, the descriptions and definitions of the present application shall prevail.

While the present invention has been described in connection with contents that are considered to be exemplary specific embodiments, it should be understood that the present invention is not limited to the disclosed specific embodiments, but is intended to cover various arrangements contained in the most broadly interpreted spirit and scopes in order to cover all such modifications and equivalent arrangements.

1. A composition for improving cognitive abilities, which consists essentially of royal jelly, Taiwan green propolis extract and soybean lecithin, as active components.

2. The composition of claim 1, which consists of royal jelly, Taiwan green propolis extract and soybean lecithin, as active components.

3. The composition of claim 1, which further comprises auxiliary ingredients: Gastrodia elata extract, black soybean powder, and soybean germ powder.

4. The composition of claim 1, wherein the royal jelly contains decenoic acid in the content of more than 3% by the weight.

5. The composition of claim 1, wherein the Taiwan green propolis extract contains isoprenoid flavonoids in the contents of more than 8% by the weight.

6. The composition of claim 1, wherein the soybean lecithin contains phosphatidylserine in the content of more than 70% by the weight.

7. The composition of claim 1, wherein the composition comprises more than 1% of decenoic acid, more than 2% of isoprenoid flavonoids and more than 1% of phosphatidylserine by the weight of the composition.

8. The composition of claim 1, which comprises 135 to 750 grams of royal jelly, 100 to 600 grams of Taiwan green propolis extract, 10 to 70 grams of soybean lecithin, 18 to 240 grams of black soybean powder, 15 to 125 grams of Gastrodia elata extract and 55 to 325 grams of soybean germ powder per 1000 grams by the total weight of the composition.

9. The composition of claim 7, which further comprises 18 to 240 grams of black soybean powder, 15 to 125 grams of Gastrodia elata extract and 55 to 325 grams of soybean germ powder per 1000 grams of the composition.

10. The composition of claim 8, which comprises main active components consisting essentially of 270 to 500 grams of royal jelly, 200 to 400 grams of Taiwan green propolis extract, 20 to 45 grams of soybean lecithin, 36 to 160 grams of black soybean powder, 30 to 82 grams of Gastrodia elata extract and 115 to 215 grams of soybean germ powder per 1000 grams of the composition.

11. The composition of claim 1, which comprises main active components consisting essentially of 338 to 420 grams of royal jelly, 253 to 315 grams of Taiwan green propolis extract, 26 to 36 grams of soybean lecithin, 45 to 134 grams of black soybean powder, 41 to 68 grams of Gastrodia elata extract and 143 to 178 grams of soybean germ powder per 1000 grams of the composition.

12. The composition of claim 1, which is in the form of a nutriment supplement, a health food or a healthcare food.

13. The composition of claim 1, wherein the composition is in the form of a medicated food or a drug.

14. The composition of claim 1, wherein the cognitive abilities comprise memory, judgment, intuition, calculation and reaction abilities.

15. The composition of claim 1, wherein the composition improves cognitive abilities of a subject.

16. The composition of claim 13, wherein the composition improves intuition, calculation and reaction abilities of a subject.

17. The composition of claim 1, further comprising abilities to resist memory degradation and to prevent dementia, effects on prevention of decline or damages in brain cell functions, activation of brain cells, improvement of memory, learning ability and attention of a subject, clear thinking, and which is absorbed well by the subject.

18.-20. (canceled)
